(19) United States
(12) Patent Application Publication
(10) Pub. No.: US 2012/0010254 A1
(43) Pub. Date: Jan. 12, 2012

(54) COMPOUNDS AND METHODS FOR TREATMENT OF INFLUENZA

(75) Inventors: Brian Mario Pinto, Coquitlam (CA); Sankar Mohan, Burnaby (CA)

(73) Assignee: Simon Fraser University, Burnaby (CA)

(21) Appl. No.: 13/142,750

(22) PCT Filed: Dec. 30, 2009
(86) PCT No.: PCT/CA09/01907

§ 371 Date: Sep. 23, 2011

Related U.S. Application Data
(60) Provisional application No. 61/193,856, filed on Dec. 30, 2008.

(51) Int. Cl. A61K 31/4192; C12Q 1/70; A61K 31/196; A61P 31/16; C07D 249/04; C07C 61/22

(52) U.S. Cl. 514/359; 548/255; 562/507; 514/563; 435/5

(57) ABSTRACT

The present invention provides in part a compound of Formula (I) or a pharmaceutically-acceptable salt or stereoisomer thereof: where R₁ is selected from the group consisting of a substituted triazole group, a guanidine group, a urea group, a thiourea group, an amidine group, and N₃; and R₂ is selected from the group consisting of H, Me, Et and an amino acid, and methods and uses thereof.

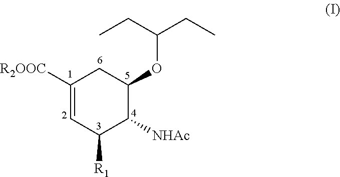

### FIELD OF INVENTION

The present invention relates to compounds and methods for treating influenza. More specifically, the invention provides compounds and methods for inhibiting influenza virus Type A neuraminidases.

### BACKGROUND OF THE INVENTION

Influenza type A and B viral infections continue to be one of the serious health problems facing the human population worldwide.

The outer membrane of the influenza virion is made up of a lipid bilayer that is densely studded with two viral surface glycoproteins: (i) hemagglutinin (HA); and (ii) neuraminidase (NA). Both these proteins are virulence factors: HA binds to host-cells via sialic acid residues, which facilitates penetration; and NA cleaves sialic acid residues from the viral envelope to facilitate release of progeny viruses, thus catalyzing the release of newly formed virions from the infected cells.

Type A influenza viruses—identified as being responsible for pandemics—are differentiated by their HA (one of 16) and NA (one of 9) subtypes. The HA1/NA1 subtype (H1N1) caused the “Spanish Flu” of 1918-1919 and resulted in as many as 50 million deaths worldwide. The H1N1 and H3N2 subtypes circulating today are much less virulent but are highly contagious, causing seasonal influenzas. In addition to the seasonal reappearance of the previously circulated viral strains, the appearance of new viral strains, such as the swine flu virus (H1N1), through antigenic variation has resulted in three major pandemics during the past 100 years. In addition, the H5N1 of avian influenza virus (AIV) is both highly transmissible and virulent and has repeatedly jumped the species barrier to kill several hundred people. Should H5N1 evolve to become more easily transmissible among humans, or H1N1 or H3N₂ undergo an antigenic shift (reassortment of genes among animal and human influenza viruses) where the HA gene is replaced by a completely new HA gene, further influenza pandemics will likely occur.

The phylogenetic tree divides the nine known neuraminidase subtypes from influenza A into two groups: group-1 contains N1, N4, N5 and N8 subtypes whereas group-2 contains N2, N3, N6, N7 and N9.⁸ The X-ray crystal structure of neuraminidase contains several large well-defined pockets.¹⁷ The structure of a sialic acid (Neu5Ac2en)/neuraminidase complex reveals that sialic acid binds to the enzyme in a considerably deformed conformation due to the strong ionic interactions of the sialic acid carboxyl moiety with the Arg118, 292 and 371 triad. The double bond of Neu5Ac2en constrains the pyranose ring into a planar structure around the ring oxygen. Three major binding pockets are presented in the neuraminidase active site. Pocket 1 with highly polar residues Glu 276, Glu 277, Arg 292, Asn 294 and hydrophobic Ala 246 has nonpolar interactions with cyclohexene-based neuraminidase inhibitors, achieving high binding affinity. Also, the active site contains a well-formed hydrophobic pocket (pocket 2), consisting of the highly conserved amino acid residues Ile 222, Arg 224 and Ala 246, which is not utilized by sialic acid for binding.¹⁷ Pocket 3 (Glu 119, Asp151, Arg 152, Trp 178, Ser 179, Ile 222 and Glu 227) is large and contains hydrophobic and hydrophilic residues. The C-4 hydroxyl and the C-5 N-acetyl groups of sialic acid bind but do not fully interact with all residues in the pocket¹⁷.

Inhibition of NA has been used in the design of anti-viral drugs.¹⁻⁴ Knowledge of the catalytic mechanism for the neuraminidase cleavage of sialic acid from glycoconjugates has resulted in the design and synthesis of analogues based on the dihydropyran ring and the cyclohexene ring. The cyclohexene series of inhibitors takes advantage of pocket 2 and forms favorable hydrophobic interactions with the conserved residues. Carbocyclic influenza neuraminidase inhibitors include a compound represented by formula (A) that is a 30 nM inhibitor of H1N1.¹⁷

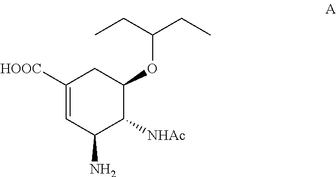

Two NA inhibitors, zanamivir³ (Relenza™) and oseltamivir,⁴ (Tamiflu™) are currently in clinical use for the treatment of influenza viral infections (Chart 1).

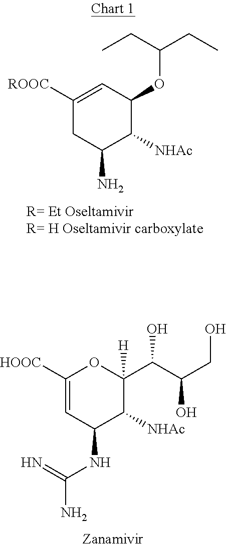

Synthesis of triazole-modified zanamivir analogues via click chemistry and anti-avian influenza virus infection activities disclosed in Li et al.¹², has resulted in the discovery of an inhibitor with a comparable EC₅₀ value (6.4 μM) against avian influenza virus H5N1 to that of the parent compound, zanamivir (2.8 μM) as shown in formula (B)¹².

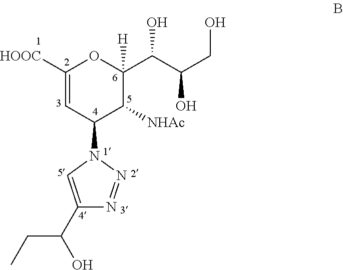

Prior to 2006, crystal structures of only two subtypes N2 and N9 from group-2 enzymes were available. In 2006, Russell et al.⁹ reported the crystal structures of three subtypes, N1, N4 and N8, from group-1 enzymes. They found that the three dimensional shapes of their active sites are virtually identical, but, interestingly, are very different when compared to those of group-2 enzymes, N2 and N9. In the case of N1, N4 and N8 subtypes (group-1), a loop of amino acids consisting of residues 147-152 (also known as loop-150), including the active site residues Asp 151 and Glu 119, was found to adopt an unusual open-conformation compared to the N2 and N9 subtypes (group-2) in which the loop-150 was found to have a closed-conformation. As a result of this open-loop conformation, a cavity near the active-site (also called cavity-150) becomes accessible in the case of N1, N4 and N8 subtypes. Oseltamivir carboxylate bound to the N1 (group-1) subtype showed similar interactions as with the group-2 enzymes, except that loop-150 adopted the open conformation as seen in the apo forms of group-1 enzymes; however, under certain crystallization conditions, loop-150 adopted the closed conformation as seen in apo and holo forms of group-2 enzymes, suggesting a slow loop closure upon inhibitor binding. The observation of two different active-site conformations between certain subtypes of group-1 and group-2 enzymes suggested that these two groups are not only genetically distinct but also structurally distinct. ⁹ Recent computational studies of the N1 subtype also suggest that loop-150 has remarkable mobility and may even open to a greater extent than observed in the crystal structures^{10,11}.

The worldwide stockpiling of zanamivir and oseltamivir as part of pandemic preparedness highlights the overall importance of neuraminidase inhibitors. The alarming threat of a potential influenza pandemic posed by the avian influenza virus H5N1^{5,6} and the recent isolations of oseltamivir-resistant H5N1 underscore the increased demand for the development of new anti-viral drugs.⁷

### SUMMARY OF THE INVENTION

The present invention provides, in part, a compound of Formula (I) or a pharmaceutically-acceptable salt or stereoisomer thereof:

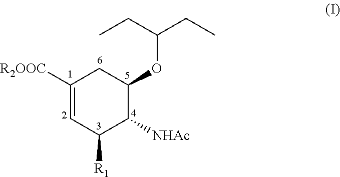

where R₁ is selected from the group consisting of a substituted triazole group, a guanidine group, a urea group, a thiourea group, an amidine group, or N₃; and R₂ is selected from the group consisting of H, Me, Et or an amino acid, such as arginine. R₁ may be a substituted 1, 2, 3-triazole group. The compound may be one or more of the compounds described herein for example compounds 1 to 7, 39, 43 or 44. The compound may be a prodrug.

The compound may inhibit an influenza virus Type A group-1 neuraminidase. The inhibition may be selective.

In alternative aspects, the invention provides a pharmaceutical composition comprising a compound as described herein in combination with a pharmaceutically acceptable carrier.

In alternative aspects, the invention provides a method of inhibiting an influenza virus Type A group-1 neuraminidase in a subject (e.g., a human) in need thereof, the method comprising administering to the subject an effective amount of a compound of Formula (I) or prodrug or a pharmaceutically acceptable salt thereof, as described herein. The inhibiting may be selective.

In alternative aspects, the invention provides a method of treating or preventing an influenza virus Type A infection in a subject (e.g., a human) in need thereof, comprising administering to the subject an effective amount of a compound of Formula (I) or prodrug or pharmaceutically acceptable salt thereof, as described herein.

In alternative aspects, the invention provides use of a compound of an effective amount of a compound of Formula (I) or prodrug or a pharmaceutically acceptable salt thereof, as described herein, in the preparation of a medicament. The medicament may be for inhibiting an influenza virus Type A group-1 neuraminidase. The medicament may also be for selectively inhibiting an influenza virus Type A group-1 neuraminidase.

In alternative aspects, the invention provides a method for screening for a selective inhibitor of an influenza virus group-1 neuraminidase, by contacting a first sample with a test compound; contacting a second sample with a compound of Formula (I) as described herein and determining the level of inhibition of the influenza virus group-1 neuraminidase in the first and second samples, where the test compound is a selective inhibitor of an influenza virus group-1 neuraminidase if the test compound exhibits the same or greater inhibition of the influenza virus group-1 neuraminidase when compared to the compound of Formula (I).

In alternative aspects, the invention provides a method of making a composition for inhibiting an influenza virus Type A group-1 neuraminidase, the method comprising admixing an effective amount of a compound of Formula (I) as described herein, with a pharmaceutically acceptable carrier. The inhibiting may be selective.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a molecular model of the active site complex of the N1 subtype with oseltamivir carboxylate, showing the C-5 amino group as a potential modification site (left panel). The chemical structure of oseltamivir carboxylate, with the carbon atoms on the cyclohexene ring numbered, is presented in the right panel.

FIGS. 2A-B show 1D traces of ¹H NMR spectra of compounds 32 (FIG. A) and 32(D) (FIG. B), with the chemical structures of the compounds shown in the right panels.

### DETAILED DESCRIPTION

The invention provides, in part, compounds which inhibit influenza virus Type A neuraminidase. The invention also provides methods of synthesizing such compounds and uses thereof e.g., for treatment of influenza Type A infection.

In some aspects, the invention provides a compound for inhibiting an influenza virus Type A neuraminidase, intermediates of the compound, prodrugs of the compound, uses of the compounds, intermediates and the prodrugs, pharmaceutical compositions including the compounds or prodrugs of the compounds, and methods of treating infections caused by influenza viruses. By “inhibits,” “inhibition” or “inhibiting” means a decrease in influenza virus Type A neuraminidase activity or biological function in the presence of a compound according to the invention, or a known inhibitor of an influenza virus Type A neuraminidase, by at least about 10% to at least about 100%, or any value therebetween, for example about 25%, 30%, 35%, 40%, 45%, 50%, 55%, 60%, 65%, 70%, 75%, 80%, 85%, 90%, 95%, 99%, or 100% when compared to the influenza virus Type A neuraminidase activity or biological function in the absence of the compound or known inhibitor. In alternative embodiments, by “inhibits,” “inhibition” or “inhibiting” is meant a decrease in influenza virus Type A neuraminidase activity or biological function in the presence of a compound according to the invention, or a known inhibitor of an influenza virus Type A neuraminidase, by at least about 1-fold or more, for example, about 1.5-fold to about 100-fold, or any value therebetween for example about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when compared to the influenza virus Type A neuraminidase activity or biological function in the absence of the compound or known inhibitor. Inhibition may be determined using standard techniques as known in the art or described herein. It is to be understood that the inhibition does not require full inhibition.

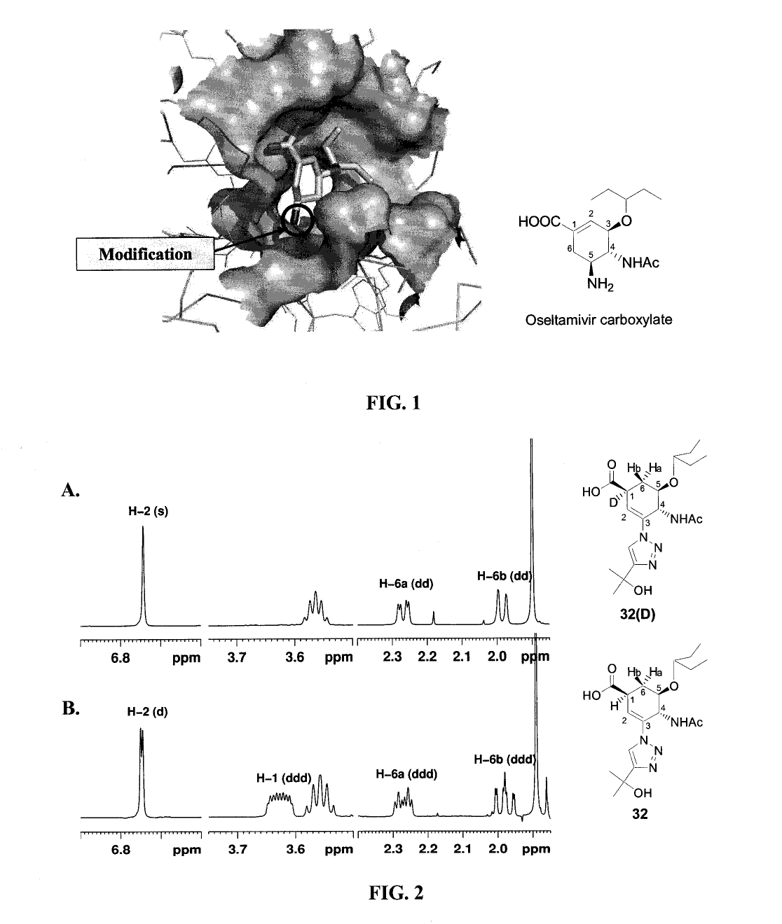

Examination of the crystal structure of oseltamivir carboxylate bound in the N1 subtype indicated that the C-5 amino group of oseltamivir carboxylate is well exposed towards the newly discovered cavity (FIG. 1). Accordingly, the C-5 amino group was used as a modification site in the design of Type A group-1 specific inhibitors.

In some embodiments, the invention provides Type A group-1 specific neuraminidase inhibitors that exhibit selectivity in inhibiting an influenza virus Type A group-1 neuraminidase. Without being bound to any particular theory, these inhibitors may provide interactions in loop-150 to fill the cavity and keep the mobile loop 150 in an energetically-favorable open conformation. In some embodiments, the compounds according to the invention are more selective for an influenza virus Type A group-1 neuraminidase over an influenza virus Type A group-2 neuraminidase. A compound that “selectively” inhibits an influenza virus Type A group-1 neuraminidase is a compound that inhibits the activity or biological function of an influenza virus Type A group-1 neuraminidase, but does not substantially inhibit the activity or biological function of a non-influenza virus group-1 neuraminidase, such as an influenza virus group-2 neuraminidase. In some embodiments, a selective inhibitor of an influenza virus Type A group-1 neuraminidase selectively binds to an influenza virus Type A group-1 neuraminidase.

By an “influenza virus group-1 neuraminidase” is meant a neuraminidase that has been categorized as belonging to the N1, N4, N5 or N8 subtypes.⁸ In alternative embodiments, by an “influenza virus group-1 neuraminidase” is meant a neuraminidase having an active site that is capable of adopting a three-dimensional configuration substantially similar to those of the N1, N4 or N8 subtypes as reported in Russell et al. ⁹. By an “influenza virus group-2 neuraminidase” is meant a neuraminidase that has been categorized as belonging to the N2, N3, N6, N7 and N9 subtypes.⁸ In alternative embodiments, by an “influenza virus group-2 neuraminidase” is meant a neuraminidase having an active site that is capable of adopting a three-dimensional configuration substantially similar to those of the N2, N3, N6, N7 or N9 subtypes as reported in Russell et al.⁹.

In one aspect, the invention provides a compound of Formula (I) or a salt, intermediate, prodrug or stereoisomeric form thereof:

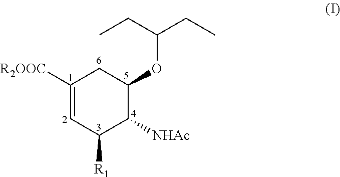

As set forth in Formula (I), R₁ may be a substituted triazole group, a guanidine group, a urea group, a thiourea group, an amidine group, or may be N₃ and R₂ may be H, Me, Et or an amino acid.

In some embodiments, R₁ may be a substituted 1, 2, 3 triazole group. In some embodiments, R₂ may be arginine.

In some embodiments, the triazole substituents may be alkyl or may be aryl.

“Alkyl” refers to a straight or branched hydrocarbon chain group consisting solely of carbon and hydrogen atoms, containing no unsaturation and including, for example, from one to ten or more carbon atoms, and which is attached to the rest of the molecule by a single bond. Unless stated otherwise specifically in the specification, the alkyl group may be optionally substituted. Unless stated otherwise specifically herein, it is understood that the substitution can occur on any carbon of the alkyl group. In some embodiments, the triazole substituents include a branched or unbranched 3-10 carbon (e.g., 3, 4, 5, 6, 7, 8, 9, or 10) alkyl chain attached directly to the triazole ring and optionally substituted with a hydroxyl or amino group on the first carbon.

“Aryl” refers to a phenyl or naphthyl group, including for example, 5 to 12 members. Unless stated otherwise specifically herein, the term “aryl” is meant to include aryl groups optionally substituted by one or more substituents as described herein, for example aryl or alkyl substituents.

In one embodiment, and without being bound to any particular theory, triazole-containing carbocycles are synthesized in which a C-4′ substituent of the triazole ring may serve as the cavity filling group and a rigid triazole ring may serve as a linker between the cavity filling group and an oseltamivir-like scaffold.

In another embodiment, the invention provides compounds having the general formula II:

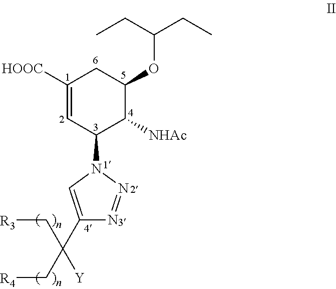

where R₃ may be alkyl or aryl, R₄ may be alkyl or aryl, n may be 1, 2, 3, 4, 5, 6, 7, 8, 9, or 10, and Y may be OH or NH₂.

In a further embodiment, the invention provides compounds having the general formula III:

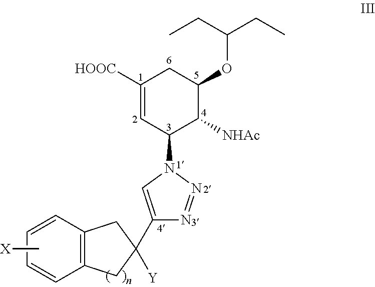

where n may be 1, 2, or 3, Y may be OH or NH₂ and X may be alkyl, aryl, OH, OMe, or NH₂.

In alternative embodiments, neuraminidase inhibitors according to the invention have the general chemical formula (IV).

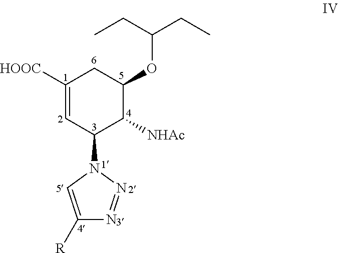

where R may be as set forth in Chart 2.

In specific embodiments, neuraminidase inhibitors according to the invention have the chemical structures as indicated by compounds 1-7 and 45-49 (Chart 2).

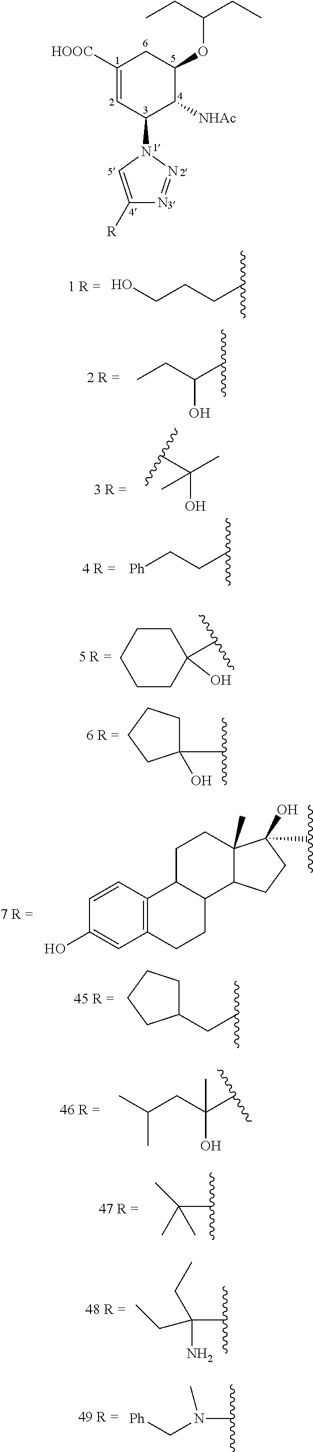

In alternative embodiments, neuraminidase inhibitors according to the invention have the chemical structure (V).

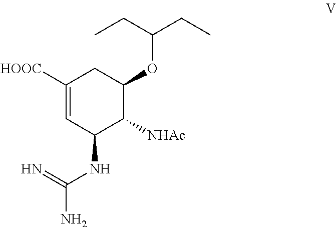

In alternative embodiments, neuraminidase inhibitors according to the invention have the chemical structure (VI).

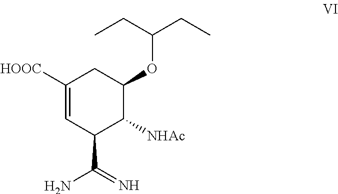

In alternative embodiments, neuraminidase inhibitors according to the invention have the chemical structure (VII).

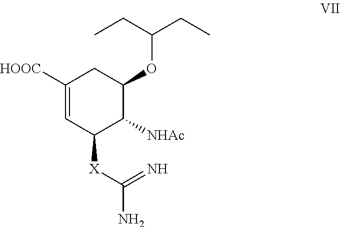

where X may be O or S.

Throughout this application, it is contemplated that the term “compound” or “compounds” refers to the compounds discussed herein as compounds according to the invention (e.g., compounds according to Formulae (I) to (VII) or as described in Chart 2) and includes precursors, intermediates and derivatives, e.g., synthetic derivatives, of the compounds, and pharmaceutically acceptable salts of the compounds, precursors, intermediates and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

In some embodiments, compounds according to the invention may be substantially pure. A compound is “substantially pure” when it is separated from the components that accompany it during the process of chemical synthesis. Typically, a compound is substantially pure when it is at least 10%, 20%, 30%, 40%, 50%, or 60%, more generally 70%, 75%, 80%, or 85%, or over 90%, 95%, or 99% by weight, of the total material in a sample.

In some embodiments, the compounds of the invention contain at least one chiral center. In some embodiments, the formulations, preparations, and compositions including compounds according to the invention include mixtures of stereoisomers, individual stereoisomers, and enantiomeric mixtures, and mixtures of multiple stereoisomers. In general, the compound may be supplied in any desired degree of chiral purity. In alternative embodiments, a compound according to the invention comprises a single stereoisomer.

The compounds according to the invention may be useful for treating influenza in a subject.

Pharmaceutical & Veterinary Compositions, Dosages, and Administration

Pharmaceutical and/or veterinary compositions including compounds according to the invention, or for use according to the invention, are contemplated as being within the scope of the invention. In some embodiments, pharmaceutical and/or veterinary compositions including an effective amount of a compound of Formula (I) are provided. It is to be understood that pharmaceutical compositions and uses include compositions for veterinary use.

In some embodiments, one or more of the compounds of formula (I) and their pharmaceutically acceptable salts, stereoisomers, solvates, and derivatives are useful because they have pharmacological activity in animals, including humans. In some embodiments, the compounds according to the invention are stable in plasma, when administered to a subject.

In some embodiments, compounds according to the invention, or for use according to the invention, may be provided in combination with any other active agents or pharmaceutical and/or veterinary compositions, where such combined therapy is useful to treat influenza (e.g., influenza Type A and/or B) or other indication. In some embodiments, compounds according to the invention, or for use according to the invention, may be provided in combination with one or more agents useful in the prevention or treatment of influenza (e.g., influenza Type A and/or B). Examples of such agents include, without limitation, oseltamivir, zanamivir, peramivir, amantadine, and/or rimantadine.

It is to be understood that combination of compounds according to the invention, or for use according to the invention, with influenza treatment agents is not limited to the examples described herein, but includes combination with any agent useful for the treatment of influenza (e.g., influenza Type A and/or B) or other indication. Combinations of compounds according to the invention, or for use according to the invention, and other influenza or other treatment agents may be administered separately or in conjunction. The administration of one agent may be prior to, concurrent to, or subsequent to the administration of other agent(s).

In alternative embodiments, the compounds of the invention may be supplied as “prodrugs” or protected forms, which release the compound after administration to a subject. For example, the compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. In some embodiments, the prodrug compound may offer advantages of solubility, tissue compatibility or delayed release in a subject.

The term “prodrug” is also meant to include any covalently bonded carriers which release the active compound of the invention in vivo when such prodrug is administered to a subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and acetamide, formamide, and benzamide derivatives of amine functional groups in the compounds of the invention and the like.

A discussion of prodrugs may be found in “Smith and Williams' Introduction to the Principles of Drug Design,” H. J. Smith, Wright, Second Edition, London (1988); Bundgard, H., Design of Prodrugs (1985), pp. 7-9, 21-24 (Elsevier, Amsterdam); The Practice of Medicinal Chemistry, Camille G. Wermuth et al., Ch 31, (Academic Press, 1996); A Textbook of Drug Design and Development, P. Krogsgaard-Larson and H. Bundgaard, eds. Ch 5, pgs 113 191 (Harwood Academic Publishers, 1991); Higuchi, T., et al., “Pro-drugs as Novel Delivery Systems,” A.C.S. Symposium Series, Vol. 14; or in Bioreversible Carriers in Drug Design, ed. Edward B. Roche, American Pharmaceutical Association and Pergamon Press, 1987, all of which are incorporated in full by reference herein.

Suitable prodrug forms of the compounds of the invention include without limitation embodiments in which, for example, R₂ of Formula I is optionally substituted alkyl, alkenyl, alkynyl, aryl, or heteroaryl. In these cases the ester groups may be hydrolyzed in vivo (e.g. in bodily fluids), releasing the active compounds in which R₂ of Formula I is H. Alternative prodrug embodiments of the invention are the compounds of Formula (I) where R₂ is CH₃, for example, as exemplified in compounds 23-29 herein, or is CH₂CH₃. In alternative embodiments, suitable prodrug forms of the compounds of the invention include without limitation embodiments where R₂ of Formula I is an amino acid moiety, for example, as exemplified in compounds 43 and 44 herein.

Compounds according to the invention, or for use according to the invention, can be provided alone or in combination with other compounds in the presence of a liposome, an adjuvant, or any pharmaceutically acceptable carrier, diluent or excipient, in a form suitable for administration to a subject. If desired, treatment with a compound according to the invention may be combined with more traditional and existing therapies for the therapeutic indications described herein. Compounds according to the invention may be provided chronically or intermittently. “Chronic” administration refers to administration of the compound(s) in a continuous mode as opposed to an acute mode, so as to maintain the initial therapeutic effect (activity) for an extended period of time. “Intermittent” administration is treatment that is not consecutively done without interruption, but rather is cyclic in nature. The terms “administration,” “administrable,” or “administering” as used herein should be understood to mean providing a compound of the invention to the subject in need of treatment.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, inhalant or emulsifier that has been approved, for example, by the United States Food and Drug Administration or other governmental agency as being acceptable for use in humans or animals. It is to be understood that a pharmaceutically acceptable carrier, diluent or excipient includes those for veterinary uses.

The compounds of the present invention may be administered in the form of pharmaceutically acceptable salts. In such cases, pharmaceutical compositions in accordance with this invention may comprise a salt of such a compound, preferably a physiologically acceptable salt, which are known in the art. In some embodiments, the term “pharmaceutically acceptable salt” as used herein means an active ingredient comprising compounds according to the invention, e.g., Formula I, used in the form of a salt thereof, for example where the salt form may confer on the active ingredient improved pharmacokinetic properties as compared to the free form of the active ingredient or other previously disclosed salt form.

A “pharmaceutically acceptable salt” includes both acid and base addition salts. A “pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

A “pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Thus, the term “pharmaceutically acceptable salt” encompasses acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of the compounds of the present invention can be used as a dosage for modifying solubility or hydrolysis characteristics, or can be used in sustained release or prodrug formulations. Also, pharmaceutically acceptable salts of the compounds of this invention may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

Pharmaceutical formulations will typically include one or more carriers acceptable for the mode of administration of the preparation, be it by injection, inhalation, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The table or capsule may be enteric coated, or in a formulation for sustained release. Many suitable formulations are known, including, polymeric or protein microparticles encapsulating a compound to be released, ointments, gels, hydrogels, or solutions which can be used topically or locally to administer a compound. A sustained release patch or implant may be employed to provide release over a prolonged period of time. Many techniques known to skilled practitioners are described in Remington: the Science &Practice of Pharmacy by Alfonso Gennaro, 20^{th} ed., Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The compounds or pharmaceutical compositions according to the present invention may be administered by oral or non-oral, e.g., intramuscular, intraperitoneal, intravenous, intracisternal injection or infusion, subcutaneous injection, inhalation, transdermal or transmucosal routes. In some embodiments, compounds or pharmaceutical compositions in accordance with this invention or for use in this invention may be administered by means of a medical device or appliance such as an inhaler, implant, graft, prosthesis, stent, etc. Implants may be devised which are intended to contain and release such compounds or compositions. An example would be an implant made of a polymeric material adapted to release the compound over a period of time. The compounds may be administered alone or as a mixture with a pharmaceutically acceptable carrier e.g., as solid formulations such as tablets, capsules, granules, powders, etc.; liquid formulations such as syrups, injections, etc.; injections, drops, suppositories, pessaries. In some embodiments, compounds or pharmaceutical compositions in accordance with this invention or for use in this invention may be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

The compounds of the invention may be used to treat animals, including mice, rats, horses, cattle, sheep, dogs, cats, pigs, and monkeys. However, compounds of the invention can also be used in other organisms, such as avian species (e.g., chickens, turkeys, geese, etc.). The compounds of the invention may also be effective for use in humans. The term “subject” or alternatively referred to herein as “patient” is intended to be referred to an animal, preferably a mammal, most preferably a human, who has been the object of treatment, observation or experiment. However, the compounds, methods and pharmaceutical compositions of the present invention may be used in the treatment of animals. Accordingly, as used herein, a “subject” may be a human, non-human primate, rat, mouse, cow, horse, pig, sheep, goat, dog, cat, etc.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as inhibition of an influenza virus Type A neuraminidase e.g., an influenza virus Type A group-1 neuraminidase, or treatment of influenza. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as inhibition of an influenza virus Type A neuraminidase e.g., an influenza virus Type A group-1 neuraminidase, or prevention of development of influenza. Typically, a prophylactic dose is used in subjects prior to or at an earlier stage of disease, so that a prophylactically effective amount may be less than a therapeutically effective amount. A suitable range for therapeutically or prophylactically effective amounts of a compound may be any value from 0.1 nM-0.1M, 0.1 nM-0.05M, 0.05 nM-15 μM or 0.01 nM-10 μM.

In alternative embodiments, in the treatment or prevention of conditions which require modulation of influenza virus group-1 neuraminidase activity, an appropriate dosage level will generally be about 0.01 to 500 mg per kg subject body weight per day, and can be administered in singe or multiple doses. In some embodiments, the dosage level will be about 0.1 to about 250 mg/kg per day. It will be understood that the specific dose level and frequency of dosage for any particular patient may be varied and will depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. In general, compounds of the invention should be used without causing substantial toxicity, and as described herein, the compounds exhibit a suitable safety profile for therapeutic use. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Other Uses and Assays

A compound of Formula (I) may be used in screening assays for compounds which modulate the activity of neuraminidases, for example, an influenza virus Type A neuraminidase. The ability of a test compound to inhibit an influenza virus Type A neuraminidase-dependent release of an influenza progeny virus, or to inhibit a virus-like particle, may be measured using any assays, as described herein or known to one of ordinary skill in the art. For example, a fluorescence or UV-based assay known in the art may be used. A “test compound” is any naturally-occurring or artificially-derived chemical compound. Test compounds may include, without limitation, peptides, polypeptides, synthesised organic molecules, naturally occurring organic molecules, and nucleic acid molecules. A test compound can “compete” with a known compound such as a compound of Formula (I) by, for example, interfering with inhibition of an influenza virus Type A neuraminidase-dependent release of an influenza progeny virus or by or by inhibiting a virus-like particle. The influenza virus Type A neuraminidase may be an influenza virus Type A group-1 neuraminidase.

Generally, a test compound can exhibit any value between 10% and 200%, or over 500%, modulation when compared to a compound of Formula (I) or other reference compound. For example, a test compound may exhibit at least any positive or negative integer from 10% to 200% modulation, or at least any positive or negative integer from 30% to 150% modulation, or at least any positive or negative integer from 60% to 100% modulation, or any positive or negative integer over 100% modulation. A compound that is a negative modulator will in general decrease modulation relative to a known compound, while a compound that is a positive modulator will in general increase modulation relative to a known compound.

In general, test compounds are identified from large libraries of both natural products or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla., USA), and PharmaMar, Mass., USA. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to modulate inhibition of influenza virus Type A neuraminidase-dependent release of an influenza progeny virus, or to inhibit a virus-like particle, further fractionation of the positive lead extract is necessary to isolate chemical constituents responsible for the observed effect. Thus, the goal of the extraction, fractionation, and purification process is the careful characterization and identification of a chemical entity within the crude extract having an influenza virus Type A neuraminidase inhibitory activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic, prophylactic, diagnostic, or other value may be subsequently analyzed using a suitable animal model or human volunteers.

Various alternative embodiments and examples of the invention are described herein. These embodiments and examples are illustrative and should not be construed as limiting the scope of the invention.

### EXAMPLES

### Example 1

### General Methods

NMR/MS: ¹H and ¹³C NMR spectra were recorded at 600 and 150 MHz, respectively. All assignments were confirmed with the aid of two-dimensional ¹H, ¹H (COSY) and/or ¹H, ¹³C(HSQC) experiments using standard pulse programs. Processing of the spectra was performed with MestRec and/or MestReNova software. Analytical thin-layer chromatography (TLC) was performed on aluminum plates precoated with silica gel 60F-254 as the adsorbent. The developed plates were air-dried, exposed to UV light and/or sprayed with a solution containing 1% Ce(SO₄)₂ and 1.5% molybdic acid in 10% aqueous H₂SO₄, and heated. Column chromatography was performed with Silica gel 60 (230-400 mesh). High resolution mass spectra were obtained by the electrospray ionization method, using an Agilent 6210 TOF LC/MS high resolution magnetic sector mass spectrometer.

Influenza virus sialidase (N1) activity assay: In a standard 96-well plate format, using previously described virus-like particles (VLP) that contain an influenza virus N1 activity¹⁸, the synthesized compounds were assayed for their capacity to inhibit influenza virus sialidase (N1) by a modification¹⁹ of the fluorometric method of Potier et al²⁰ using the fluorogenic substrate 4-methylumbelliferyl N-acetyl-α-D-neuraminide (MUN).

Specifically, 7 μA, of 50 mM sodium acetate-6 mM CaCl₂ buffer (pH 5.5) was added to each well of a 96-well solid black plate on ice, followed by 1 μL of inhibitor, 1 μL of N1-containing VLP, and finally 1 μl, of the substrate MUN. The plate was then briefly centrifuged up to 1000 rpm for approx 10 s to combine all components and the reaction was incubated at 37° C. with 900 rpm shaking for 20 min. To stop the reaction, 250 μL 0.25 M glycine pH 10 was added to each well and the fluorescence was read (1 sec per well) at an excitation of 355 nm and emission of 460 nm. All inhibition assays were done in triplicate over four inhibitor concentrations and at two concentrations of the substrate MUN (0.05 mM and 0.1 mM). Inhibitor concentrations were selected to give a percentage inhibition of sialidase activity between 10 and 90% and data analysis was carried out using SigmaPlot Enzyme Kinetics Module.²¹

### Example 2

### Synthesis of Compounds

Target carbocycles (1-7) were synthesized from the azido intermediate 9 and various terminal alkynes via click chemistry, as described below (Scheme 1).

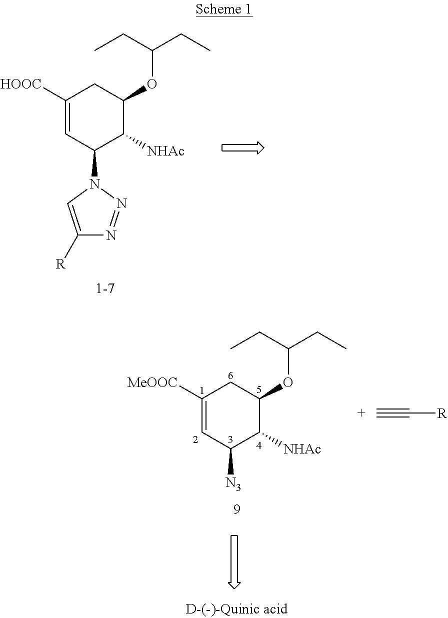

The preparation of the acetonide intermediate 10 was achieved in a two-step sequence starting from D-(−)-quinic acid, as shown in Scheme 2, using a literature procedure.¹³

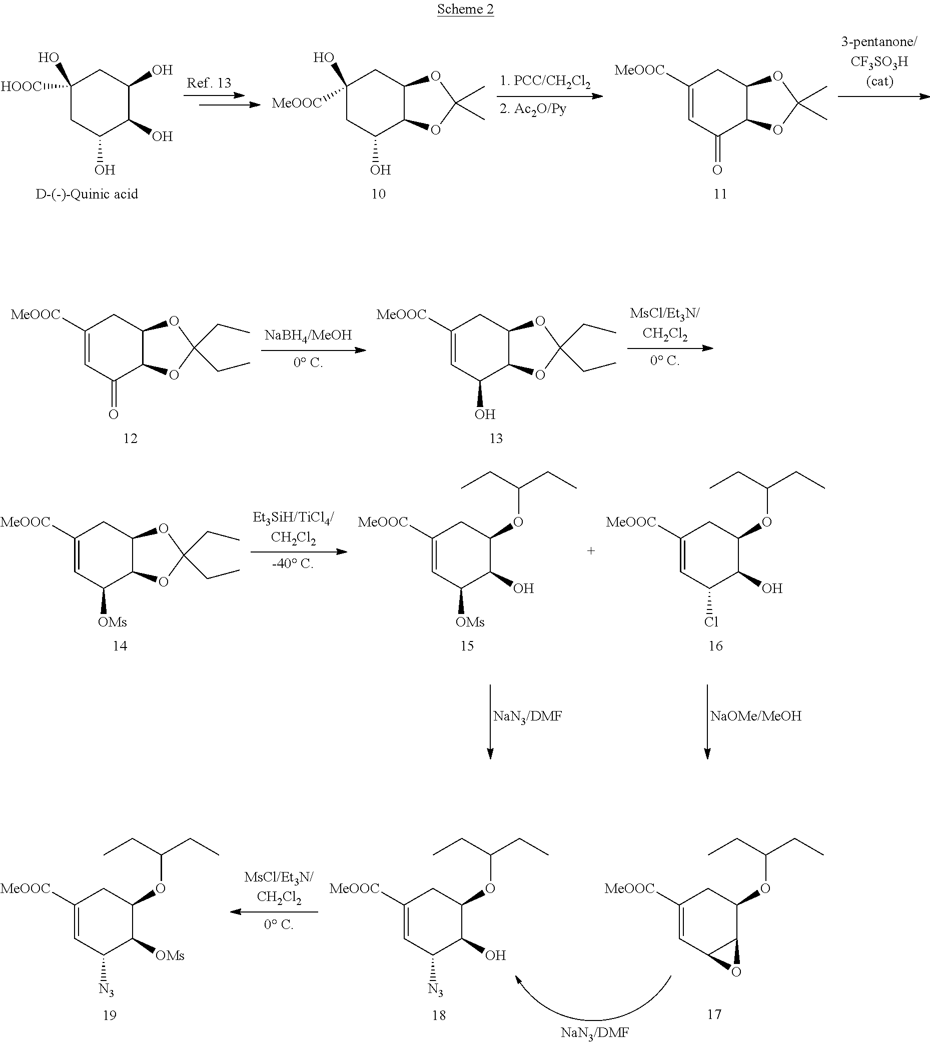

Oxidation of 10 using PCC-alumina was patterned after a literature method¹⁴ in which the oxidation of the same compound, but with a different acetal protecting group, was reported. However, we found that with our intermediate 10, after the addition of PCC-alumina, the reaction mixture turned into a tarry thick mass and as a result, uniform stirring was not possible. The reported processing procedure involves dilution of the reaction mixture with dichloromethane followed by filtration and concentration of the filtrate to get the crude product. In our case, a tarry black mass was obtained even after two filtrations; consequently, purification of the crude product became difficult and as a result a low yield of the product was obtained (55%). Hence, we modified the work-up procedure as follows: after the reaction had gone to completion (as indicated by TLC), the solvent was removed and the crude mass was suspended in diethyl ether and stirred for 2 h. After filtration, the solid residue was resuspended in diethyl ether, stirred for 1 h, and filtered again. The filtrates were combined and concentrated to give the crude product without any contamination of the black mass and also in higher yields.

The crude product was then reacted with acetic anhydride and excess pyridine to give the enone 11 in 75% yields for two steps. Trans-ketalization of 11 was accomplished using a large excess of 3-pentanone and a catalytic amount of trifluoromethane sulfonic acid to give the transketalized intermediate 12, which was then reduced in a regioselective manner using NaBH₄ at 0° C. to give the allylic alcohol 13 in 80% yield. The intermediate 13 was then transformed into the mesylate 14 using MsCl and triethylamine. The mesylate 14, upon reaction with Et₃SiH and TiCl₄ at −40° C., underwent regioselective, reductive ring opening to give the desired secondary alcohol 15 in 89% yield.

An interesting side product was also formed in this reaction which was isolated (10% yield) and characterized as the chloride intermediate 16. We predicted the stereochemistry at C-3 in the chloride 16 as R and the anti-relationship between the C-3 chloro substituent and the C-4 hydroxyl group based on the assumption that the chloride might have formed via S_{N}2 displacement of the mesyl group in 15. The proposed stereochemistry at C-3 was confirmed later by the successful conversion of the chloride intermediate into an epoxide (17) by reaction with NaOMe. The mesylate 15 was transformed into the azido alcohol 18 using sodium azide. Of note, the side product 16 in the previous step was also transformed into the desired azido intermediate 18 via a two step process, namely, reaction with NaOMe followed by reaction with sodium azide, as shown in Scheme 2. The azido alcohol 18 was then reacted with MsCl and triethylamine to give the mesylate 19 in 85% yield.

Reduction of the azido group in 19 using PPh₃ and triethylamine/water mixture resulted in the formation of the aziridine compound 20, as shown in Scheme 3.

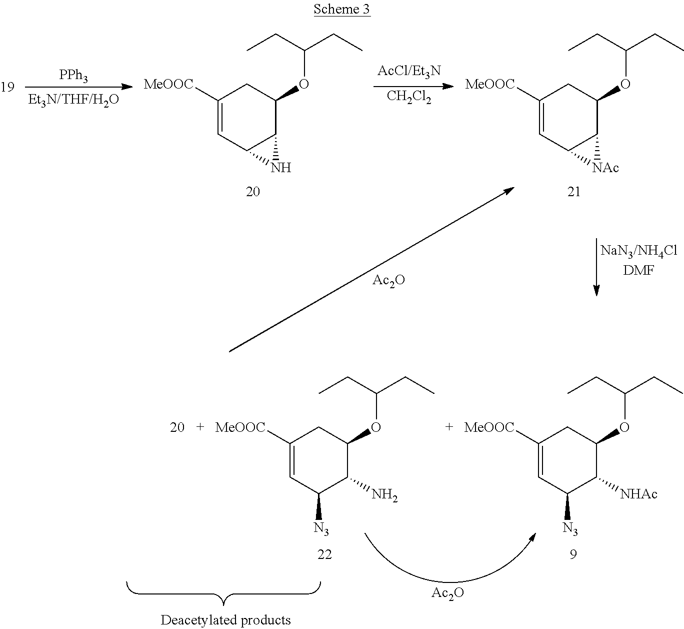

The intermediate 20, upon reaction with acetyl chloride and triethylamine at 0° C., gave the N-acetyl aziridine 21 in 75% yield. Reaction of 21 with sodium azide and NH₄Cl gave a mixture of three products, as indicated by TLC (Scheme 3). The major product was isolated (60% yield) and characterized as the desired azido intermediate 9. The other two side products could not be separated by column chromatography. However, we anticipated that these two side products could have been formed as a result of the deacetylation of both starting material 21 and product 9. Hence, the reaction was repeated and when the starting material was fully consumed (as indicated by TLC), the reaction was stopped and after processing, the crude product was treated with acetic anhydride at RT for 2 h. TLC of the reaction mixture indicated only two spots corresponding to starting material 20 and product 9, thereby confirming that the two side products observed in the previous step were indeed the deacetylated products 20 and 22. In this case, the desired azido intermediate 9 was isolated in 68% yield and the recovered starting material 21 (20%) was then recycled.

With the key intermediate 9 in hand, we then performed a copper-catalyzed Huisgen 1,3-dipolar cycloaddition¹⁵ using various terminal alkynes by following the standard protocol, as shown in Scheme 4.

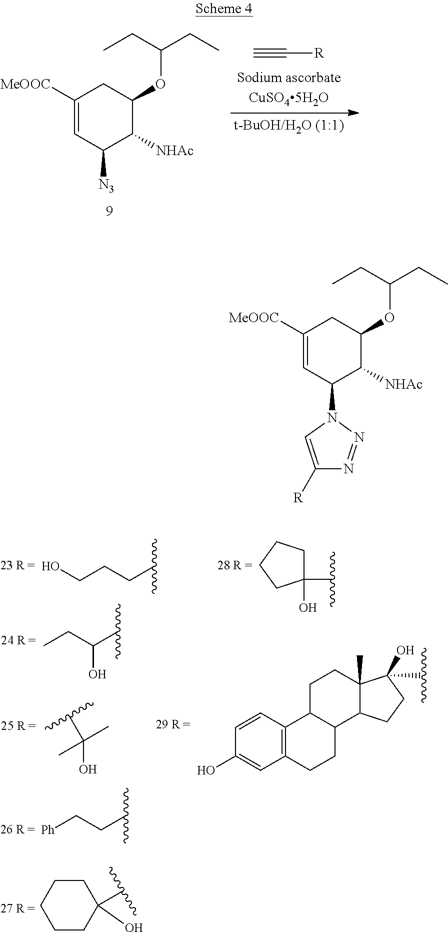

Initially, the hydrolysis of the methyl ester 25 was performed using 1M NaOH and MeOH as solvent (Scheme 5).

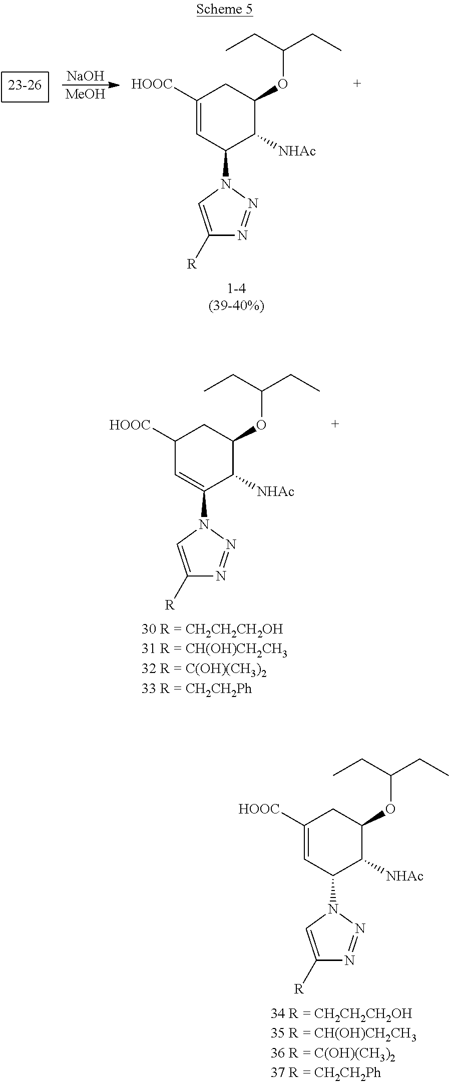

More specifically, compounds 1-4 were prepared as follows.

4-Acetylamino-5-(1-ethyl-propoxy)-3-[4-(3-hydroxy-propyl)-[1,2,3]triazol-1-yl]-cyclohex-1-enecarboxylic acid (Compound 1): NaOH (32 mg, 0.8 mmol) was added to a solution of the methyl ester 23 (65 mg, 0.16 mmol) in a 1:1 mixture of MeOH and water (3 mL) and the reaction mixture was stirred at room temperature for 3-4 h. The pH of the reaction mixture was adjusted to 6 using 0.1N HCl, and solvents were evaporated. The crude compound was dissolved in MeOH and passed through a pad of silica gel and the filtrate was concentrated to give a pale yellow gum. Addition of ethyl acetate precipitated the desired compound 1 as a colorless powder (28 mg, 44%).

The NMR and MS data were as follows: ¹H NMR (CD₃OD): δ 7.76 (1H, s, H-5′), 6.70 (1H, br t, H-2), 5.51 (1H, br d, J_{3,4}=9.0 Hz, H-3), 4.19 (1H, dd, J_{4,5}=9.6 Hz, H-4), 3.89 (1H, dt, J_{5,6a}=5.4, J_{5,6b}=9.0 Hz, H-5), 3.57 (2H, t, J=6.6 Hz, HOCH₂CH₂CH₂—), 3.40 (1H, m, J=6.0 Hz, (CH₃CH₂)₂CH—O—), 3.01 (1H, br dd, J_{6a,6b}=18 Hz, H-6a), 2.76 (2H, t, J=7.8 Hz, HOCH₂CH₂CH₂—), 2.39 (1H, ddt, J_{6b,2}=J_{6b,3}=3.0 Hz, H-6b), 1.87 (2H, m, J=7.2 Hz, HOCH₂CH₂CH₂—), 1.84 (3H, s, —NHCOCH₃), 1.56-1.45 (4H, m, (CH₃CH₂)₂CH—O—), 0.92 and 0.86 (3H, t, J=7.8 Hz, (CH₃CH₂)₂CH—O—). ¹³C NMR (CD₃OD): δ 173.5 (—NHCOCH₃), 169.3 (—COOH), 149.2 (C-4′), 134.2 (C-1), 133.7 (C-2), 122.6 (C-5′), 83.0 (CH₃CH₂)₂CH—O—), 74.3 (C-5), 63.0 (C-3), 62.0 (HOCH₂CH₂CH₂—), 57.1 (C-4), 33.3 (HOCH₂CH₂CH₂—), 33.3 (C-6), 27.3 and 27.0 (CH₃CH₂)₂CH—O—), 23.0 (—NHCOCH₃), 22.9 (HOCH₂CH₂CH₂—), 9.9 and 9.8 (CH₃—CH₂)₂—CH—O—). HRMS Calcd for C₁₉H₃₁N₄O₅ (M+H): 395.2294. Found: 395.2282.

4-Acetylamino-5-(1-ethyl-propoxy)-3-[4-(1-hydroxy-propyl)-[1,2,3]triazol-1-yl]-cyclohex-1-enecarboxylic acid (Compound 2): Compound 2 (41 mg, colorless powder, 42% yield) was obtained as a 1:1 mixture of diastereomers from compound 24 (100 mg, 0.25 mmol) using the same procedure as described to obtain 1. Data for the mixture of diastereomers.

The NMR and MS data were as follows: ¹H NMR (CD₃OD): δ 7.89 and 7.87 (1H, s, H-5′), 6.76 and 6.75 (1H, br t, H-2), 5.50 (2H, br d, J_{3,4}=9.0 Hz, H-3), 4.73 and 4.72 (1H, dd, J=7.0 Hz, CH₃CH₂CH(OH)—), 4.24 and 4.22 (1H, dd, J_{4,5}=10.0 Hz, H-4), 3.91 (2H, dt, J_{5,6a}=5.5, J_{5,6b}=9.5 Hz, H-5), 3.41 (2H, m, J=5.5 Hz, (CH₃CH₂)₂CH—O—), 3.0 (2H, br dd, J_{6a,6b}=18 Hz, H-60, 2.42 (2H, ddt, J_{6b,2}=J_{6b,3}=3.5 Hz, H-6b), 1.93-1.81 (4H, m, CH₃CH₂CH(OH)—), 1.86 and 1.85 (3H, s, —NHCOCH₃), 1.59-1.47 (8H, m, (CH₃CH₂)₂CH—O—), 0.96 and 0.95 (3H, t, J=7.5 Hz, CH₃CH₂CH(OH)—), 0.94 and 0.88 (6H, t, J=7.5 Hz, (CH₃CH₂)₂CH—O—). ¹³C NMR (CD₃OD): δ 172.2 and 172.1 (—NHCOCH₃), 167.5 (—COOH), 151.9 and 151.8 (C-4′), 132.9 (C-2), 132.4 and 132.3 (C-1), 121.3 and 121.2 (C-5′), 81.7 (CH₃CH₂)₂CH—O—), 73.0 (C-5), 67.9 and 67.8 (CH₃CH₂CH(OH)—), 61.8 and 61.7 (C-3), 55.8 and 55.7 (C-4), 31.9 and 31.8 (C-6), 30.2 and 30.1 (CH₃CH₂CH(OH)—), 26.0 and 25.7 (CH₃CH₂)₂CH—O—), 21.7 (—NHCOCH₃), 9.0 and 8.9 (CH₃CH₂CH(OH)—), 8.7, 8.6 and 8.5 (CH₃—CH₂)₂—CH—O—). HRMS Calcd for C₁₉H₃₁N₄O₅ (M+H): 395.2294. Found: 395.2284.

4-Acetylamino-5-(1-ethyl-propoxy)-3-[4-(1-hydroxy-1-methyl-ethyl)-[1,2,3]triazol-1-yl]-cyclohex-1-enecarboxylic acid (Compound 3): Compound 3 (78 mg, colorless powder, 40% yield) was obtained from compound 25 (200 mg, 0.49 mmol) using the same procedure as described to obtain compound 1.

The NMR and MS data were as follows: ¹H NMR (CD₃OD): δ 7.83 (1H, s, H-5′), 6.74 (1H, br t, H-2), 5.53 (1H, br d, J_{3,4}=8.4 Hz, H-3), 4.20 (1H, dd, J_{4,5}=9.6 Hz, H-4), 3.90 (1H, dt, J_{5,6a}=5.4, J_{5,6b}=9.0 Hz, H-5), 3.39 (1H, m, J=5.4 Hz, (CH₃CH₂)₂CH—O—), 3.0 (1H, hr dd, J_{6a,6b}=18 Hz, H-6a), 2.41 (1H, ddt, J_{6b,2}=J_{6b,3}=3.0 Hz, H-6b), 1.84 (3H, s, —NHCOCH₃), 1.57 (6H, s, (CH₃)₂C(OH)—), 1.60-1.46 (4H, m, (CH₃CH₂)₂CH—O—), 0.92 and 0.86 (3H, t, J=7.8 Hz, (CH₃CH₂)₂CH—O—). ¹³C NMR (CD₃OD): δ 173.6 (—NHCOCH₃), 168.9 (—COOH), 157.5 (C-4′), 134.1 (C-2), 133.7 (C-1), 121.2 (C-5′), 83.0 (CH₃CH₂)₂CH—O—), 74.2 (C-5), 69.2 ((CH₃)₂C(OH)—), 62.9 (C-3), 57.1 (C-4), 33.0 (C-6), 30.7 ((CH₃)₂C(OH)—), 27.3 and 27.0 (CH₃CH₂)₂CH—O—), 23.0 (—NHCOCH₃), 9.9 and 9.8 (CH₃CH₂)₂CH—O—). HRMS Calcd for C₁₉H₃₁N₄O₅ (M+H): 395.2294. Found: 395.2304.

4-Acetylamino-5-(1-ethyl-propoxy)-3-(4-phenethyl-[1,2,3]triazol-1-yl)-cyclohex-1-enecarboxylic acid (Compound 4): Compound 4 (46 mg, colorless powder, 32% yield) was obtained from compound 26 (150 mg, 0.37 mmol) using the same procedure as described to obtain compound 1.

The NMR and MS data were as follows: ¹H NMR (CD₃OD): δ 7.62 (1H, s, H-5′), 7.29-7.14 (5H, m, Ar), 6.66 (1H, br t, H-2), 5.50 (1H, br d, J_{3,4}=9.6 Hz, H-3), 4.18 (1H, dd, J_{4,5}=9.6 Hz, H-4), 3.87 (1H, dt, J_{5,6a}=5.4, J_{5,6b}=9.6 Hz, H-5), 3.38 (1H, m, J=6.0 Hz, (CH₃CH₂)₂CH—O—), 3.03-2.89 (5H, m, H-6a, PhCH₂CH₂—), 2.38 (1H, ddt, J_{6b,6a}=18.0, J_{6b,2}=J_{6b,3}=3.0 Hz, H-6b), 1.83 (3H, s, —NHCOCH₃), 1.55-1.45 (4H, m, (CH₃CH₂)₂CH—O—), 0.91 and 0.86 (3H, t, J=7.8 Hz, (CH₃CH₂)₂CH—O—). ¹³C NMR (CD₃OD): δ 173.5 (—NHCOCH₃), 168.8 (—COOH), 148.9 (C-4′), 142.50, 129.7, 129.6 and 127.3 (Ar), 134.3 (C-2), 133.6 (C-1), 122.7 (C-5′), 83.0 (CH₃CH₂)₂CH—O—), 74.3 (C-5), 62.8 (C-3), 57.0 (C-4), 36.7 (PhCH₂CH₂—), 33.1 (C-6), 28.6 (PhCH₂CH₂—), 27.3 and 27.0 (CH₃CH₂)₂CH—O—), 23.0 (—NHCOCH₃), 9.9 and 9.8 (CH₃CH₂)₂CH—O—). HRMS Calcd for C₂₄H₃₃N₄O₄ (M+H): 441.2502. Found: 441.2517.

Surprisingly, the ¹H NMR spectrum of the crude product indicated three different products. Fortunately, the desired compound 3 was conveniently precipitated (40% yield, 98% purity) by the addition of ethyl acetate to the crude product. From the ethyl acetate soluble fraction, we were able to isolate one of the side products (41% yield, 96% purity) by crystallization and it was then characterized as being 32 by 1D and 2D NMR analyses. From the remaining mother liquor, the other side product was isolated (13% yield) as a mixture containing 15% of 32, and characterized as being 36. Similar results were obtained for the hydrolysis of the other esters, 23, 24 and 26.

We attribute the formation of 30-33 and 34-37 to the base-catalyzed double bond migration and epimerization at the C-3 stereocenter, respectively, as shown for compound 25 in Scheme 6.

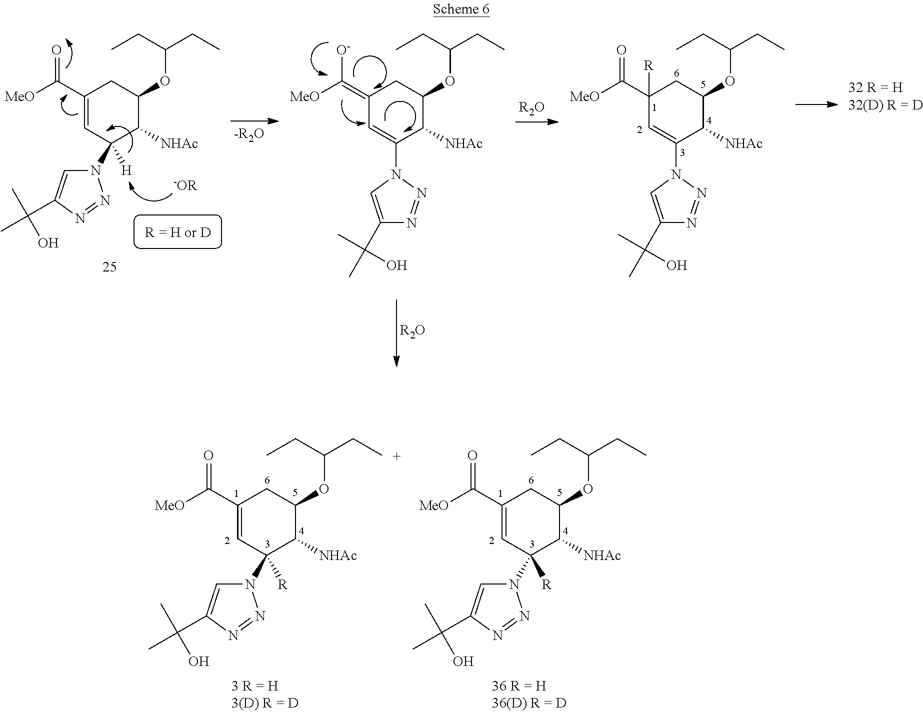

In an attempt to provide further evidence for the proposed mechanism, we have performed the hydrolysis of compound 25 using deuterated sodium hydroxide in deuterated methanol. As expected, deuterium was incorporated in the isolated products, 3(D), 32 (D), and 36(D) at the C-3, C-1, and C-3 positions, respectively, as indicated by ¹H NMR analyses; the spectrum of compound 32 (D) lacked the H-1 signal (FIG. 2B) when compared to 32 (FIG. 2A); similarly, the spectra of compounds 3(D) and 36(D) lacked the H-3 signals, thus indicating that deuterium incorporation had occurred at C-3 in both of these compounds.

In addition, the splitting patterns of the adjacent protons indicated the incorporation of deuterium. For example, the ¹H NMR spectrum of 32 showed a doublet for H-2 at 6.74 ppm and a ddd for both H-6a and H-6b at 2.26 ppm and 1.98 ppm (FIG. 2A), respectively, whereas in the case of 32(D), the spectrum showed a singlet for H-2 and a dd for both the H-6a and H-6b protons (FIG. 2B). Similarly, the triplet corresponding to the H-4 proton observed in the ¹H NMR spectrum of 3 (δ 4.19 ppm) and 36 (δ 4.53 ppm) was changed into a doublet in the spectra of the respective deuterated products, 3(D) and 36(D). In a reaction performed in an NMR tube, the ratio of the three products did not change even after 3 days, indicating that the double bond migration and epimerization at the C-3 stereocenter must be occurring prior to the hydrolysis of the ester group.

To overcome the difficulty of epimerization at C-3 and double bond migration, we employed trimethyltin hydroxide¹⁶ to hydrolyze the methyl esters 27-29. The reactions proceeded smoothly and the products were obtained in higher yields, as shown Scheme 7.

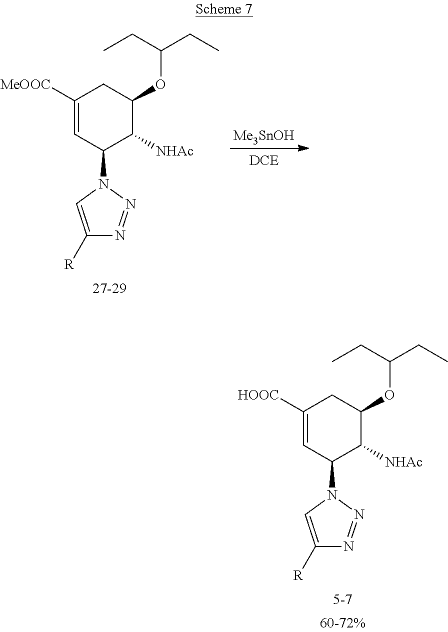

More specifically, compounds 5-7 were prepared as follows.

4-Acetylamino-5-(1-ethyl-propoxy)-3-[4-(1-hydroxy-cyclohexyl)-[1,2,3]triazol-1-yl]-cyclohex-1-enecarboxylic acid (Compound 5): A mixture of compound 27 (137 mg, 0.31 mmol) and trimethyltin hydroxide (442 mg, 2.45 mmol) in 8 mL of dichloroethane (DCE) was heated at 80° C. for 5 h. Solvents were removed under reduced pressure and the crude mass was purified by column chromatography (EtOAc:MeOH, 7:3 (v/v)). Compound 5 was obtained as a colorless foam (80 mg, 61%).

The NMR and MS data were as follows: ¹H NMR (CD₃OD): δ 7.85 (1H, s, H-5′), 6.71 (1H, br t, H-2), 5.52 (1H, br d, J_{3,4}=9.0 Hz, H-3), 4.22 (1H, dd, J_{4,5}=9.6 Hz, H-4), 3.89 (1H, dt, J_{5,6a}=6.0, J_{5,6b}=9.6 Hz, H-5), 3.40 (1H, m, J=5.4 Hz, (CH₃CH₂)₂CH—O—), 3.01 (1H, br dd, J_{6a,6b}=18 Hz, H-6a), 2.41 (1H, ddt, J_{6b,2}=J_{6b,3}=3.5 Hz, H-6b), 2.04-1.98 (2H, m), 1.84 (3H, s, —NHCOCH₃), 1.83-1.72 (4H, m), 1.61-1.56 (7H, m, (CH₃CH₂)₂CH—O—), 1.42-1.36 (1H, m), 0.92 and 0.86 (3H, t, J=7.8 Hz, (CH₃CH₂)₂CH—O—). ¹³C NMR (CD₃OD): δ 173.5 (—NHCOCH₃), 169.4 (—COOH), 157.1 (C-4′), 134.4 (C-1), 133.5 (C-2), 121.8 (C-5′), 83.0 (CH₃CH₂)₂CH—O—), 74.3 (C-5), 70.4 (1C), 63.0 (C-3), 57.1 (C-4), 39.0 and 38.9 (2C), 33.2 (C-6), 27.3 and 27.0 (CH₃CH₂)₂CH—O—), 26.7 (1C), 23.3 and 23.2 (2C), 23.0 (—NHCOCH₃), 9.9 and 9.8 (CH₃CH₂)₂CH—O—). HRMS Calcd for C₂₂H₃₅N₄O₅ (M+H): 435.2607. Found: 435.2611.

4-Acetylamino-5-(1-ethyl-propoxy)-3-[4-(1-hydroxy-cyclopentyl)-[1,2,3]triazol-1-yl]-cyclohex-1-enecarboxylic acid (Compound 6): Compound 6 (63 mg, 62%, colorless powder) was obtained from compound 28 (106 mg, 0.24 mmol) using the same procedure as described to obtain 5.

The NMR and MS data were as follows: ¹H NMR (CD₃OD): δ 7.84 (1H, s, H-5′), 6.74 (1H, br t, H-2), 5.54 (1H, br d, J_{3,4}=9.0 Hz, H-3), 4.20 (1H, dd, J_{4,5}=9.6 Hz, H-4), 3.90 (1H, dt, J_{5,6a}=5.4, J_{5,6b}=9.6 Hz, H-5), 3.39 (1H, m, J=5.4 Hz, (CH₃CH₂)₂CH—O—), 2.99 (1H, br dd, J_{6a,6b}=18 Hz, H-6a), 2.41 (1H, ddt, J_{6b,2}=J_{6b,3}=3.5 Hz, H-6b), 2.12-2.04 (2H, m), 1.98-1.89 (4H, m), 1.84 (3H, s, —NHCOCH₃), 1.82-1.77 (2H, m), 1.57-1.45 (4H, m, (CH₃CH₂)₂CH—O—), 0.92 and 0.86 (3H, t, J=7.8 Hz, (CH₃CH₂)₂CH—O—). ¹³C NMR (CD₃OD): δ 173.5 (—NHCOCH₃), 168.9 (—COOH), 156.0 (C-4′), 134.1 (C-2), 133.7 (C-1), 121.6 (C-5′), 82.9 (CH₃CH₂)₂CH—O—), 79.6 (1C), 74.5 (C-5), 63.1 (C-3), 57.2 (C-4), 41.9 and 41.8 (2C), 33.6 (C-6), 27.3 and 27.0 (CH₃CH₂)₂CH—O—), 24.6 (2C), 23.0 (—NHCOCH₃), 10.0 and 9.8 (CH₃CH₂)₂CH—O—). HRMS Calcd for C₂₁H₃₃N₄O₅ (M+H): 421.2451. Found: 421.2454.

4-Acetylamino-3-[4-((17α)-estra-1,3,5(10)-triene-3,17-dihydroxy-17-yl)-[1,2,3]triazol-1-yl]-5-(1-ethyl-propoxy)-cyclohex-1-enecarboxylic acid (Compound 7): Compound 7 (79 mg, 72%, yellow foam) was obtained from compound 29 (115 mg, 0.18 mmol) using the same procedure as described to obtain 5.

The NMR and MS data were as follows: ¹H NMR (CD₃OD): δ 7.78 (1H, s, H-5′), 6.99 (1H, d, J=8.4 Hz, Ar), 6.78 (1H, br t, H-2), 6.50 (1H, br dd, J=8.4 Hz, Ar), 6.45 (1H, d, J=2.4 Hz, Ar), 5.53 (1H, br d, J_{3,4}=9.6 Hz, H-3), 4.26 (1H, dd, J_{4,5}=10.0 Hz, H4), 3.87 (1H, dt, J_{5,6a}=5.4, J_{5,6b}=9.6 Hz, H-5), 3.38 (1H, m, J=6.0 Hz, (CH₃CH₂)₂CH—O—), 3.01 (1H, br dd, J_{6a,6b}=18.0 Hz, H-6a), 2.80-2.70 (2H, m), 2.46-2.41 (1H, m), 2.40 (1H, ddt, J_{6b,2}=J_{6b,3}=3.5 Hz, H-6b), 2.16-1.88 (5H, m), 1.85 (3H, s, —NHCOCH₃), 1.64-1.59 (2H, m), 1.56-1.45 (5H, m, (CH₃CH₂)₂CH—O—), 1.44-1.27 (3H, m), 1.03 (3H, s), 0.91 and 0.86 (3H, t, J=7.8 Hz, (CH₃CH₂)₂CH—O—), 0.66 (1H, m). ¹³C NMR (CD₃OD): δ 173.3 (—NHCOCH₃), 168.8 (—COOH), 156.0 (1C, Ar), 155.6 (C-4′), 139.0 (1C, Ar), 134.6 (C-2), 133.3 (C-1), 132.7 and 127.3 (2C, Ar), 123.9 (C-5′), 116.2 and 113.8 (2C, Ar), 83.3 (CH₃CH₂)₂CH—O—), 83.0 (1C), 74.4 (C-5), 62.9 (C-3), 57.2 (C-4), 49.5, 48.5, 41.2, 38.6 and 34.4 (5C), 33.4 (C-6), 30.9, 28.8, 27.7, 27.3, 27.0 and 24.8 (7C), 23.2 (—NHCOCH₃), 15.0 (1C), 9.9 and 9.8 (CH₃CH₂)₂CH—O—). HRMS Calcd for C₃₄H₄₇N₄O₆ (M+H): 607.3496. Found: 607.3516.

Finally, the key intermediate 9 was also converted into the double bond isomer of oseltamivir, 38 and its corresponding guanidine derivative 39, as shown in Scheme 8.

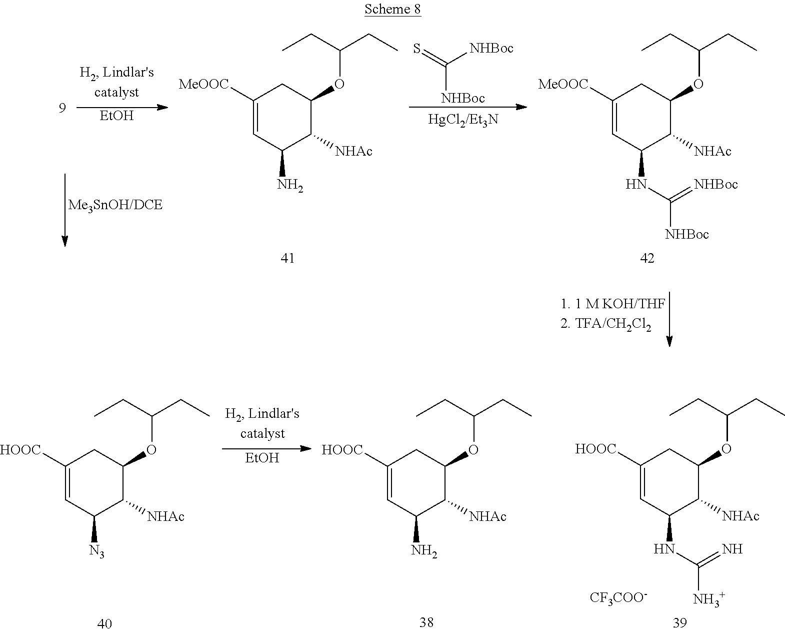

More specifically, compounds 38 and 39 were prepared as follows.

4-Acetylamino-3-amino-5-(1-ethyl-propoxy)-cyclohex-1-enecarboxylic acid (Compound 38): Hydrogen gas was bubbled through a mixture of compound 40 (20 mg, 0.06 mmol) and Lindlar's catalyst (8 mg) in 4 mL of ethanol at room temperature for 2.5 h. The reaction mixture was filtered through Celite and the filtrate was concentrated and purified by column chromatography (EtOAc/MeOH/H₂O, 2:3:0.1 (v/v)). Compound 38 was obtained as yellow foam (14 mg, 82% yield).

The NMR and MS data were as follows: ¹H NMR (D₂O): δ 6.29 (1H, br t, H-2), 4.07 (1H, dd, J_{4,3}=J_{4,5}=9.6 Hz, H-4), 4.0 (1H, br d, H-3), 3.80 (1H, dt, J_{5,6a}=5.4, J_{5,6b}=9.6 Hz, H-5), 3.48 (1H, m, J=5.4 Hz, (CH₃CH₂)₂CH—O—), 2.89 (1H, br dd, J_{6a,6b}=17.4 Hz, H-6a), 2.33 (1H, ddt, J_{6b,2}=J_{6b,3}=3.0 Hz, H-6b), 2.06 (3H, s, —NHCOCH₃), 1.59-1.44 (4H, m, (CH₃CH₂)₂CH—O—), 0.86 (6H, t, J=7.8 Hz, (CH₃CH₂)₂CH—O—). ¹³C NMR (D₂O): δ 174.9 (—NHCOCH₃), 173.8 (—COOH), 138.7 (C-1), 125.0 (C-2), 83.3 (CH₃CH₂)₂CH—O—), 73.4 (C-5), 52.7 (C-4), 52.6 (C-3), 32.3 (C-6), 25.3 (CH₃CH₂)₂CH—O—), 22.3 (—NHCOCH₃), 8.7 and 8.4 (CH₃CH₂)₂CH—O—). HRMS Calcd for C₁₄H₂₅N₂O₄ (M+H): 285.1814. Found: 285.1813.

4-Acetylamino-5-(1-ethyl-propoxy)-3-guanidino-cyclohex-1-enecarboxylic acid Trifluoroacetate salt (Compound 39): 1N KOH (2.1 mL) was added to a solution of compound 42 (70 mg, 0.13 mmol) in 7 mL of THF and the reaction mixture was stirred at room temperature for 28 h. The pH of the reaction mixture was adjusted to 7 by bubbling CO₂ and the solvents were evaporated. The crude mass was purified by column chromatography (EtOAc/MeOH, 9:1 (v/v)) and the resulting carboxylic acid (53 mg) was then dissolved in 1:1 mixture of TFA and dichloromethane (2 mL) and stirred at room temperature for 2 h. Solvents were evaporated and the crude mass was then washed with dichloromethane (2×2 mL) to yield compound 39 as a colorless foam (29 mg, 51% yield).

The NMR and MS data were as follows: ¹H NMR (D₂O): δ 6.70 (1H, dd, J_{2,3}=J_{2,6a}=2.4 Hz, H-2), 4.38 (1H, br d, J_{3,4}=9.0 Hz, H-3), 4.07 (1H, dd, J_{4,5}=9.0 Hz, H-4), 3.85 (1H, dt, J_{5,6a}=5.4, J_{5,6b}=9.0 Hz, H-5), 3.50 (1H, m, J=5.4 Hz, (CH₃CH₂)₂CH—O—), 2.95 (1H, br dd, J_{6a,6b}=18 Hz, H-6a), 2.33 (1H, ddt, J_{6b,2}=J_{6b,3}=3.5 Hz, H-6b), 2.03 (3H, s, —NHCOCH₃), 1.60-1.43 (4H, m, (CH₃CH₂)₂CH—O—), 0.87 and 0.86 (3H, t, J=7.8 Hz, (CH₃CH₂)₂CH—O—). ¹³C NMR (D₂O): δ 174.6 (—NHCOCH₃), 169.3 (—COOH), 162.9 (q, J_{C,F}=141 Hz, CF₃COO⁻), 156.8 (NH₂C(NH)—NH—), 135.2 (C-2), 130.5 (C-1), 116.3 (q, J_{C,F}=1160 Hz, CF₃COO⁻), 83.4 (CH₃CH₂)₂CH—O—), 73.1 (C-5), 54.4 (C-4), 53.6 (C-3), 31.2 (C-6), 25.4 and 25.3 (CH₃CH₂)₂CH—O—), 22.1 (—NHCOCH₃), 8.7 and 8.2 (CH₃CH₂)₂CH—O—). HRMS Calcd for C₁₇H₂₇F₃N₄O₆ (M—CF₃COO⁻): 327.2032. Found: 327.2031.

We note that compound 38 is a known 30 nM inhibitor of neuraminidase.¹⁷ Thus, intermediate 9, upon treatment with trimethyltin hydroxide, gave intermediate 40 which upon treatment with Lindlar's catalyst gave the amine 38. On the other hand, intermediate 9 upon treatment with Lindlar's catalyst gave compound 41 which was then converted into the Boc-protected guanidine derivative 42 using Boc-protected thiourea and HgCl₂, as shown in Scheme 8. The methyl ester 42 was first hydrolyzed using 1M KOH and then the Boc protecting groups were removed using a 1:1 mixture of TFA and CH₂Cl₂ to yield compound 39.

Due to the insoluble nature of compounds 4 and 7 in water, the corresponding L-arginine salts, 43 and 44 (Chart 3), respectively, were made and tested.

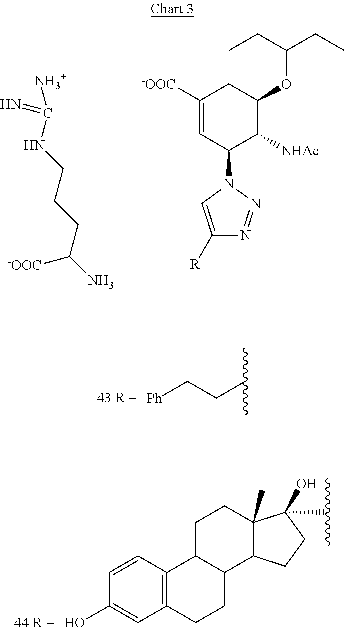

4-Acetylamino-5-(1-ethyl-propoxy)-3-(4-phenethyl-[1,2,3]triazol-1-yl)-cyclohex-1-enecarboxylic acid L-arginine salt (Compound 43): A mixture of L-arginine (4.15 mg, 0.02 mmol) and compound 4 (10.5 mg, 0.02 mmol) was stirred in dry MeOH at room temperature for 4 h. The solvents were removed and the resulting salt 43 (14 mg, hygroscopic colorless solid) was dried under vacuum.

4-Acetylamino-3-[4-((17α)-estra-1,3,5(10)-triene-3,17-dihydroxy-17-yl)-[1,2,3]triazol-1-yl]-5-(1-ethyl-propoxy)-cyclohex-1-enecarboxylic acid L-arginine salt (Compound 44): A mixture of L-arginine (2.95 mg, 0.02 mmol) and compound 7 (9.8 mg, 0.02 mmol) was stirred in dry MeOH at room temperature for 4 h. The solvents were removed and the resulting salt 44 (10.4 mg, hygroscopic brown solid) was dried under vacuum.

### Example 3

### Inhibition of Virus-Like Particles by Synthesized Compounds

We tested the inhibitory activities of compounds 1-7, 38 and 39 against virus-like particles (VLP) that contain an influenza virus N1 activity.¹⁸ The results are summarized in Table 1.

TABLE 1
Inhibitory activities of compounds 1-3, 5, 6, 38 and 39 against
virus-like particles that contain an influenza virus N1 activity

| Compound | Kᵢ (M) |
|---|---|
| 1 | 5.0 × 10⁻⁶ |
| 2 | 7.0 × 10⁻⁷ |
| 3 | 2.0 × 10⁻⁶ |
| 5 | 48^{a} |
| 6 | 110^{a} |
| 38 | 2.0 × 10⁻⁸ |
| 39 | 4.8 × 10⁻⁹ |
| 43 | 1.2 × 10⁻⁵ |
| 44 | 5.9 × 10⁻⁵ |
| 4-amino-4-deoxy- | 1.0 × 10⁻⁸ |
| Neu5Ac2en | |
| 4-deoxy-4-guanidino- | 1.4 × 10⁻¹⁰ |
| Neu5Ac2en | |
| (ZANAMIVIR) | |

^{a}IC₅₀ values in μM.

Our results indicate that the triazole modified compounds 1-3, 43 and 44 were inhibitors (Table 1), but less active than the parent compound 38, its guanidine derivative 39, or zanamivir. Compounds 5 and 6 were less effective as inhibitors. The parent compound 38 has a Kᵢ value of 20 nM against the virus-like particles, similar to the reported inhibitory value (IC₅₀=30 nM).¹⁷ However, its guanidine derivative 39 was found to be a more potent inhibitor, with a Kᵢ value of 4.8 nM; in comparison, zanamivir has a Kᵢ=0.14 nM in the same assay

### Example 4

### Synthesis of Compounds

Compounds as, for example, described in Chart 2 are synthesized from quinic acid, followed by click chemistry as described in Scheme 9²².

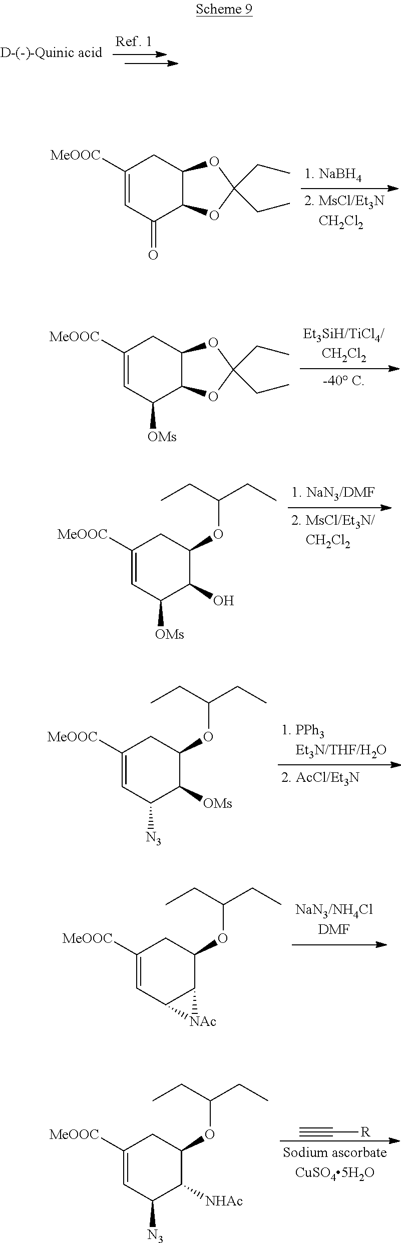

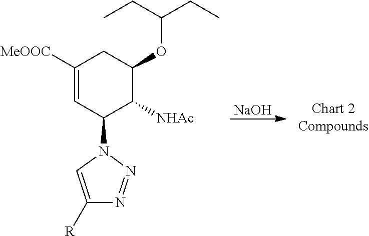

### REFERENCES

1. (a) Colman, P. M. In The influenza viruses: Influenza virus neuraminidase, Enzyme and Antigen; Krug, R. M., Ed.; Plenum Press: New York, 1989; pp 175-218. (b) Colman, P. M. Protein Sci. 1994, 3, 1687-1696.
2. Wade, R. C. Structure 1997, 5, 1139-1145.
3. (a) Taylor, N. R.; von Itzstein, M. J. Med. Chem. 1994, 37, 616. (b) von Itzstein, M.; Wu, W.-Y.; Jin, B. Carbohydr. Res. 1994, 259, 301. (c) von Itzstein, M., Wu, W. Y., Kok, G. B., Pegg, M. S., Dyason, J. C., Jin, B., Phan, T. V., Smythe, M. L., White, H. F., Oliver, S. W., Colman, P. M., Varghese, J. N., Ryan, D. M., Woods, J. M., Bethell, R. C., Hotham, V. J., Cameron, J. M., and Penn, C. R. Nature 1993, 363, 418-423.
4. Kim, C. U., Lew, W., Williams, M. A., Liu, H., Zhang, L., Swaminathan, S., Bischofberger, N., Chen, M. S., Mendel, D. B., Tai, C. Y., Layer, W. G., and Stevens, R. C. J. Am. Chem. Soc. 1997, 119, 681-690.
5. Shinya, K., Hatta, M., Yamada, S., Takada, A., Watanabe, S., Halfmann, P., Horimoto, T., Neumann, G., Kim, J. H., Lim, W., Guan, Y., Peiris, M., Kiso, M., Suzuki, T., Suzuki, Y., and Kawaoka, Y. J. Virol. 2005, 79, 9926-9932.
6. Kamps, B. S., Hoffmann, C., and Preiser, W. Influenza Report 2006, Flying Publisher, Paris, Cagliari, Wuppertal, Sevilla.
7. Le, Q. M., Kiso, M., Someya, K., Sakai, Y. T., Nguyen, T. H., Nguyen, K. H. L., Pham, N. D., Ngyen, H. H., Yamada, S., Muramoto, Y., Horimoto, T., Takada, A., Goto, H., Suzuki, T., Suzuki, Y., and Kawaoka, Y. Nature 2005, 437, 1108-1108.
8. Thompson, J. D., Higgins, D. G., and Gibson, T. J. Comput. Appl. Biosci. 1994, 10, 19-29.
9. Russell, R. J., Haire, L. F., Stevens, D. J., Collins, P. J., Lin, Y. P., Blackburn, G. M., Hay, A. J., Gamblin, S. J., and Skehel, J. J. Nature 2006, 443, 45-49.
10. Amaro, R. E., Minh, D. D., Cheng, L. S., Lindstrom, W. M. Jr., Olson, A. J., Lin, J. H., Li, W. W., and McCammon, J. A. J. Am. Chem. Soc. 2007, 129, 7764-7765.
11. Landon, M. R., Amaro, R. E., Baron, R., Ngan, C. H., Ozonoff, D., Mccammon, J. A., and Vajda, S. Chem. Biol. Drug Des. 2008, 71, 106-116.
12. Li, J., Zheng, M., Tang, W., He, L. P., Zhu, W., Li, T., Zuo, J. P., Liu, H., Jiang, H. Bioorg. Med. Chem. Lett. 2006, 16, 5009-5013.
13. Federspiel, M., Fischer, R., Hennig, M., Mair, H. J., Oberhauser, T., Rimmler, G., Albiez, T., Bruhin, J., Estermann, H., Gandert, C., Gockel, V., Gotzo, S., Hoffmann, U., Huber, G., Janatsch, G., Lauper, S., Rockel-Stabler, 0., Trussardi, R., and Zwahlen, A. G. Org. Proc. Res. Devel. 1999, 3, 266-274.
14. Ulibarri, G., Nadler, W., Skrydstrup, T., Audrain, H., Chiaroni, A., Riche, C., and Grierson, D. S. J. Org. Chem. 1995, 60, 2753-2761.
15. Huisgen, R. In 1,3-Dipolar Cycloaddition Chemistry; Padwa, A., Ed.; Wiley: Pergamon, 1984; vol. 1, pp 1-176.
16. Nicolaou, K. C., Estrada, A. A., Zak, M., Lee, S. H., Safina, B. S. Angew. Chem. Int. Ed. 2005, 44, 1378-1382.
17. Lew, W., Chen, X. W., Kim, C. U. Curr. Med. Chem. 2000, 7, 663-672.
18. Haselhorst, T., Garcia, J-M., Islam, T., Lai, J. C. C., Rose, F. J., Nicholls, J. M., Peiris, J. S. M., von Itzstein, M. Angew. Chem. Int. Ed. 2008, 47, 1910-1912.
19. Chong, A. K. J., Pegg, M. S., von Itzstein, M. Biochim. Biophys. Acta 1991, 10, 65-71.
20. Potier, M., Mameli, L., Belisle, M., Dallaire, L., Melançon, S. B. Anal. Biochem. 1979, 94, 287-296.
21. SigmaPlot Enzyme Kinetics Module, Systat Software Inc., Chicago, Ill.
22. Kok, S. H.-L., Lee, C. C., Shing, T. K. M. J. Org. Chem. 2001, 66, 7184-7190.

### OTHER EMBODIMENTS

Although various embodiments of the invention are disclosed herein, many adaptations and modifications may be made within the spirit and scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range, and of sub-ranges encompassed therein. As used herein, the terms “comprising”, “comprises”, “having” or “has” are used as an open-ended terms, substantially equivalent to the phrase “including, but not limited to”. As used herein the singular forms “a”, “and”, and “the” include plural referents unless the context clearly dictates otherwise. For example, “a compound” refers to one or more of such compounds. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A compound of Formula (I) or a pharmaceutically-acceptable salt or stereoisomer thereof: 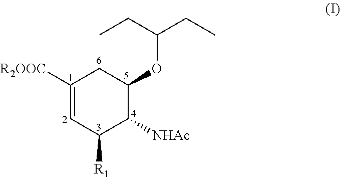
wherein
R₁ is selected from the group consisting of a substituted triazole group, a urea group, a thiourea group, an amidine group, and N₃; and
R₂ is selected from the group consisting of H, Me, Et and an amino acid.

2. The compound of claim 1 wherein R₁ is a substituted 1, 2, 3 triazole group.

3. The compound of claim 1 wherein R₂ is H.

4. The compound of claim 1 wherein R₂ is Me.

5. The compound of claim 1 wherein R₂ is arginine.

6. The compound of claim 1, wherein the compound is one or more of compounds 1 to 7, 43 or 44.

7. The compound of claim 1, wherein the compound is a prodrug.

8. The compounds of claim 1 wherein the compound inhibits an influenza virus Type A group-1 neuraminidase.

9. The compound of claim 1 wherein the compound selectively inhibits an influenza virus group-1 neuraminidase.

10. A pharmaceutical composition comprising a compound of Formula (I) or a pharmaceutically-acceptable salt or stereoisomer thereof: 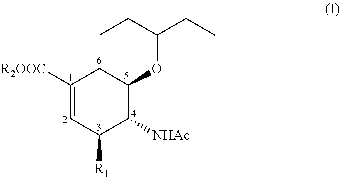
wherein
R₁ is selected from the group consisting of a substituted triazole group, a guanidine group, a urea group, a thiourea group, an amidine group, and N₃; and
R₂ is selected from the group consisting of H, Me, Et and an amino acid, in combination with a pharmaceutically acceptable carrier.

11. A method of inhibiting an influenza virus Type A group-1 neuraminidase in a subject in need thereof, the method comprising administering to the subject an effective amount of a compound of Formula (I) or prodrug or a pharmaceutically acceptable salt thereof: 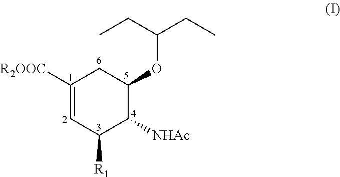
wherein
R¹ is selected from the group consisting of a substituted triazole group, a guanidine group, a urea group, a thiourea group, an amidine group, and N₃; and
R² is selected from the group consisting of H, Me, Et and an amino acid.

12. The method of claim 11 wherein the inhibiting is selective.

13. A method of treating or preventing an influenza virus Type A infection in a subject in need thereof, comprising administering to the subject an effective amount of a compound of Formula (I) or prodrug or pharmaceutically acceptable salt thereof: 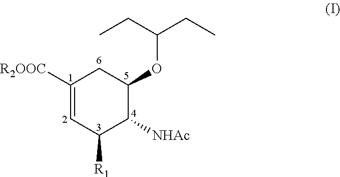
wherein
R¹ is selected from the group consisting of a substituted triazole group, a guanidine group, a urea group, a thiourea group, an amidine group, and N₃; and
R² is selected from the group consisting of H, Me, Et and an amino acid.

14. The method of any claim 11 or 13 wherein the subject is a human.

15-17. (canceled)

18. A method for screening for a selective inhibitor of an influenza virus group-1 neuraminidase, the method comprising:
a) contacting a first sample with a test compound;
b) contacting a second sample with a compound of Formula (I) 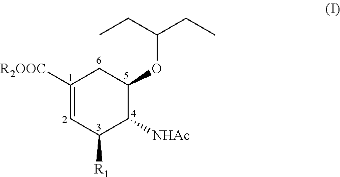
wherein
R¹ is selected from the group consisting of a substituted triazole group, a guanidine group, a urea group, a thiourea group, an amidine group, and N₃; and
R² is selected from the group consisting of H, Me, Et and an amino acid; and
c) determining the level of inhibition of the influenza virus group-1 neuraminidase in the first and second samples,
wherein the test compound is a selective inhibitor of an influenza virus group-1 neuraminidase if the test compound exhibits the same or greater inhibition of the influenza virus group-1 neuraminidase when compared to the compound of Formula (I).

19. A method of making a composition for inhibiting an influenza virus Type A group-1 neuraminidase, the method comprising admixing an effective amount of a compound of Formula (I): 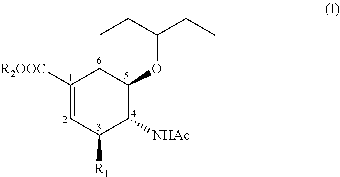
wherein
R¹ is selected from the group consisting of a substituted triazole group, a guanidine group, a urea group, a thiourea group, an amidine group, and N₃; and
R² is selected from the group consisting of H, Me, Et and an amino acid; with a pharmaceutically acceptable carrier.

20. The method of claim 19 wherein the inhibiting is selective.

21. A compound of Formula (I) or a pharmaceutically-acceptable salt or stereoisomer thereof: 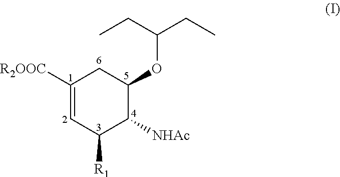
wherein
R₁ is a guanidine group; and
R₂ is selected from the group consisting of Me, Et and an amino acid.

22. The compound of claim 21 wherein R₂ is arginine.

23. The compound of claim 21, wherein the compound is a prodrug.

24. The compounds of claim 21 wherein the compound inhibits an influenza virus Type A group-1 neuraminidase.

25. The compound of claim 21 wherein the compound selectively inhibits an influenza virus group-1 neuraminidase.
